(19) United States
(12) Patent Application Publication
(10) Pub. No.: US 2024/0399015 A1
(43) Pub. Date: Dec. 5, 2024

(54) FRAGRANCE AND DISPENSER

(71) Applicant: Reckitt & Colman (Overseas) Hygiene Home Limited, Slough, Berkshire (GB)

(72) Inventors: Philip George BASSINDALE, Hull (GB); Kathryn Margaret DONNELLY, Hull (GB); Katarzyna RYBCZYNSKA, Hull (GB)

(21) Appl. No.: 18/696,090

(22) PCT Filed: Oct. 7, 2022
(86) PCT No.: PCT/GB2022/052539

§ 371 Date: Mar. 27, 2024

(30) Foreign Application Priority Data
Oct. 13, 2021 (GB) 2114622.0

(51) Int. Cl. A61L 9/14; A61L 9/01; A61L 9/12; A61L 101/34; A61L 101/36; A61L 101/38

(52) U.S. Cl. CPC A61L 9/14; A61L 9/01; A61L 9/127; A61L 2101/34; A61L 2101/36; A61L 2101/38; A61L 2209/132; A61L 2209/133

(57) ABSTRACT

The present invention provides a fragrance composition comprising at least one fragrance, at least one alkoxy alcohol solvent, and at least one co-solvent, and wherein the composition has a water content of less than 5 wt. %. The invention further provides an atomiser for dispensing a fragrance; the atomiser comprising:
a. An outer housing;
b. An orifice plate and piezo electric element;
c. A liquid reservoir comprising a liquid to be atomised, the liquid comprising the fragrance composition hereinabove; and
d. A liquid transport member,
wherein the liquid transport member extends from within the liquid reservoir to abut the orifice plate, and wherein the liquid transport member is substantially contained within the liquid reservoir and both the liquid transport member and liquid reservoir are readily detachable together from the atomiser.

### TECHNICAL FIELD OF THE INVENTION

The present invention relates to fragrance compositions comprising at least one alkoxy alcohol solvent. The invention also relates to methods of manufacturing said fragrance compositions; atomisers comprising said fragrance compositions; methods of scenting the air using said atomisers; and refills for said atomisers.

### BACKGROUND TO THE INVENTION

Mist-based delivery is a challenging format for fragrances. Often there are multiple competing considerations to account for, such as technical success, olfactive performance and regulatory compliance.

It is key for a successful fragrance composition in this format to possess the necessary physical and chemical properties to allow for good spray rate, low fallout, and good sensory performance, among other factors.

It is arguably even more important, however, that such compositions comply with safety regulations. It is further advantageous for compositions to exceed the minimum regulatory constraints and to be even safer for use.

Known compositions include those containing 75-98 wt % of a carrier solvent containing at least 50 wt % branched alkanes (isoparaffins). Whilst these compositions may deliver fragrance, such high amounts of isoparaffins can cause some toxicity issues, such as respiratory problem and skin/eye irritation.

There therefore exists a need for fragrance compositions which possess the necessary physical and chemical parameters to achieve good performance in a mist-based format, but which also allow for improved safety.

It is an aim of embodiments of the present invention to address these requirements by providing fragrance compositions which provide one or more of the following advantages:
Display good physical and chemical properties which allow for successful application in a mist-based format, preferably in conjunction with an atomiser device.
Display good spray rate, low fallout and good sensory/olfactive performance.
Display a reduced respiratory and/or aspiration hazard. Preferably contain solvents which do not carry a respiratory hazard and/or aspiration hazard classification.
Display reduced irritancy, particularly to skin and/or eyes. Preferably contain solvents which are not classed as irritants, particularly to skin and/or eyes.
Contain ingredients, and preferably solvents, which are cheap and/or readily available.

It is also an aim of embodiments of the invention to overcome or mitigate at least one problem of the prior art, whether expressly described herein or not.

### SUMMARY OF THE INVENTION

According to a first aspect of the invention, there is provided a fragrance composition comprising at least one fragrance, at least one alkoxy alcohol solvent, and at least one co-solvent, and wherein the composition has a water content of less than 5 wt. %.

“Alkoxy alcohols” are defined as compounds comprising both an alcohol moiety and an ether moiety in a single compound.

Such compositions provide good physical and chemical properties which allow for their successful application as mist-based fragrances. They possess a good spray rate, low fallout and good sensory/olfactive performance. Furthermore, such compositions display a low respiratory and aspiration hazard. They also display reduced irritancy, particularly to skin and eyes. Furthermore, alkoxy alcohol solvents are readily available, often at low cost.

Preferably, the at least one alkoxy alcohol comprises a compound of Formula (I):

R′—O—ROH (I)
wherein R′—O— is:
a straight or branched alkoxy group, optionally containing one or more rings, optionally containing one or more further heteroatoms, and/or optionally containing one or more double bonds; an aryloxy group; or an arylalkoxy group; and
R is:
a straight or branched alkyl, optionally containing one or more rings, optionally containing one or more heteroatoms, and/or optionally containing one or more double bonds; an aryl; or an aralkyl.

In some embodiments, the at least one alkoxy alcohol contains a chain of 1-10 atoms directly connecting the oxygen atom of the alkoxy group to the oxygen atom of the alcohol group. The at least one alkoxy alcohol may contain a chain of 1-9 atoms directly connecting the oxygen atom of the alkoxy group to the oxygen atom of the alcohol group, or a chain of 1-8, 1-7, 1-6, 1-5, 1-4, or a chain of 1-3 atoms directly connecting the oxygen atom of the alkoxy group to the oxygen atom of the alcohol group. In further embodiments, the at least one alkoxy alcohol may contain a chain of 2-10, 2-9, 2-8, 2-7, 2-6, 2-5, 2-4, or 2-3 atoms directly connecting the oxygen atom of the alkoxy group to the oxygen atom of the alcohol group.

There may be no greater than 20 atoms directly connecting the oxygen atom of the alkoxy group to the oxygen atom of the alcohol group, or no greater than 19, 18, 17, 16, 15, 14, 13, 12, 11, 10, 9, 8, 7, 6, or no greater than 5 atoms directly connecting the oxygen atom of the alkoxy group to the oxygen atom of the alcohol group.

In some embodiments, the chain of atoms directly connecting the oxygen atom of the alkoxy group to the oxygen atom of the alcohol comprises one or more heteroatoms, preferably oxygen atoms. There may be 1 heteroatom in the chain, or 2, 3, or 4 heteroatoms in the chain.

In preferred embodiments, the chain of atoms directly connecting the oxygen atom of the alkoxy group to the oxygen atom of the alcohol group consists entirely of carbon atoms.

References above to the chain of atoms directly connecting the oxygen atom of the alkoxy group to the oxygen atom of the alcohol group do not include atoms which may branch from the chain, but which do not form part of the direct connection of atoms between the oxygen atom of the alkoxy group and the oxygen atom of the alcohol group.

By way of example, the alkoxy alcohol of formula: R′—OCH₂C(CH₃)₂CH₂OH contains a chain of 3 atoms (all of which are carbon atoms) directly connecting the oxygen atom of the alkoxy group to the oxygen atom of the alcohol group.

In embodiments wherein R is an aryl or an aralkyl group, R may comprise an optionally substituted heteroarene, naphthalene, anthracene, or an optionally substituted benzene ring.

In preferred embodiments, R is a straight or branched alkyl, optionally containing one or more rings, optionally containing one or more heteroatoms, and/or optionally containing one or more double bonds.

In some embodiments, R is a straight or branched C₁-C₁₅, C₁-C₁₂, C₁-C₁₀, C₁-C₈, C₁-C₆, or C₁-C₈ alkyl, optionally containing one or more rings, optionally containing one or more heteroatoms, and/or optionally containing one or more double bonds.

In some embodiments, R is a straight or branched C₁-C₁₅, C₁-C₁₂, C₁-C₁₀, C₁-C₈, C₁-C₆, or C₁-C₈ alkyl.

In some embodiments, R is a straight or branched C₂-C₁₅, C₂-C₁₂, C₂-C₁₀, C₂-C₈, C₂-C₆, or C₂-C₅ alkyl, optionally containing one or more rings, optionally containing one or more heteroatoms, and/or optionally containing one or more double bonds.

In some embodiments, R is a straight or branched C₂-C₁₅, C₂-C₁₂, C₂-C₁₀, C₂-C₈, C₂-C₆, or C₂-C₅ alkyl.

In some embodiments, R is a straight or branched C₃-C₁₅, C₃-C₁₂, C₃-C₁₀, C₃-C₈, C₃-C₆, or C₃-C₅ alkyl, optionally containing one or more rings, optionally containing one or more heteroatoms, and/or optionally containing one or more double bonds.

In some embodiments, R is a straight or branched C₃-C₁₅, C₃-C₁₂, C₃-C₁₀, C₃-C₈, C₃-C₆, or C₃-C₅ alkyl.

In some embodiments, R is a straight or branched C₄-C₁₅, C₄-C₁₂, C₄-C₁₀, C₄-C₈, C₄-C₆, or C₄-C₅ alkyl, optionally containing one or more rings, optionally containing one or more heteroatoms, and/or optionally containing one or more double bonds.

In some embodiments, R is a straight or branched C₄-C₁₅, C₄-C₁₂, C₄-C₁₀, C₄-C₈, C₄-C₆, or C₄-C₅ alkyl.

In some embodiments, R is a straight or branched C₅-C₁₅, C₅-C₁₂, C₅-C₁₀, C₅-C₈, or C₅-C₆ alkyl, optionally containing one or more rings, optionally containing one or more heteroatoms, and/or optionally containing one or more double bonds.

In some preferred embodiments, R is a straight or branched C₈, C₇, C₆, C₅, C₄, C₃, C₂, or C₁ alkyl, optionally and if chemically possible containing one or two rings, optionally containing one or more heteroatoms, and/or optionally and if chemically possible containing one or more double bonds.

In some preferred embodiments, R is a straight or branched C₈, C₇, C₆, C₅, C₄, C₃, C₂, or C₁ alkyl.

In preferred embodiments, R is a straight or branched C₅ alkyl. In some particularly preferred embodiments, R is a branched C₅ alkyl, preferably 1,1-dimethylpropyl.

In preferred embodiments, R is a straight or branched C₃ alkyl. In some particularly preferred embodiments, R is a branched C₃ alkyl.

In preferred embodiments, R is a straight or branched, and preferably straight C₂ alkyl. In some particularly preferred embodiments, R is ethyl.

In some embodiments, the R group contains one or more heteroatoms, preferably oxygen atoms. The one or more heteroatoms are preferably part of the chain of atoms directly connecting the oxygen atom of the alkoxy group to the oxygen atom of the alcohol. There may be 1 heteroatom in the chain, or 2, 3, or 4 heteroatoms in the chain.

In embodiments wherein R′—O— is an aryloxy or arylalkoxy group, R′—O— may comprise an optionally substituted heteroaryloxy, naphthyloxy, anthracenyloxy, benzyloxy, or an optionally substituted phenoxy group.

In preferred embodiments, R′—O— is a straight or branched alkoxy group, optionally containing one or more rings, optionally containing one or more further heteroatoms, and/or optionally containing one or more double bonds.

In some embodiments, R′—O— is a straight or branched C₁-C₁₀, C₁-C₈, C₁-C₆, C₁-C₅, C₁-C₄, or C₁-C₃ alkoxy, optionally containing one or more rings, optionally containing one or more further heteroatoms, and/or optionally containing one or more double bonds.

In some embodiments, R′—O— is a straight or branched, and preferably straight C₁-C₁₀, C₁-C₈, C₁-C₆, C₁-C₅, C₁-C₄, or C₁-C₃ alkoxy.

In some embodiments, R′—O— is a straight or branched C₂-C₁₀, C₂-C₈, C₂-C₆, C₂-C₅, C₂-C₄, or C₂-C₃ alkoxy, optionally containing one or more rings, optionally containing one or more further heteroatoms, and/or optionally containing one or more double bonds.

In some embodiments, R′—O— is a straight or branched, and preferably straight C₂-C₁₀, C₂-C₈, C₂-C₆, C₂-C₅, C₂-C₄, or C₂-C₃ alkoxy.

In some embodiments, R′—O— is a straight or branched C₃-C₁₀, C₃-C₈, C₃-C₆, C₃-C₅, or C₃-C₄ alkoxy, optionally containing one or more rings, optionally containing one or more further heteroatoms, and/or optionally containing one or more double bonds.

In some embodiments, R′—O— is a straight or branched, and preferably straight C₃-C₁₀, C₃-C₈, C₃-C₆, C₃-C₅, or C₃-C₄ alkoxy.

In some preferred embodiments, R′—O— is a straight or branched C₅ alkoxy, optionally containing a ring, optionally containing one or more further heteroatoms, and/or optionally containing one or more double bonds.

In some preferred embodiments, R′—O— is a straight or branched, and preferably straight C₅ alkoxy.

In some preferred embodiments, R′—O— is a straight or branched C₄ alkoxy, optionally containing a ring, optionally containing one or more further heteroatoms, and/or optionally containing one or more double bonds.

In some preferred embodiments, R′—O— is a straight or branched, and preferably straight C₄ alkoxy.

In some preferred embodiments, R′—O— is a straight or branched C₃ alkoxy, optionally containing a ring, optionally containing one or more further heteroatoms, and/or optionally containing a double bond.

In some preferred embodiments, R′—O— is a straight or branched C₃ alkoxy. In some embodiments, R′—O— is isopropoxy. In preferred embodiments, R′—O— is n-propoxy.

In some preferred embodiments, R′—O— is C₂ alkoxy, optionally containing a double bond, and/or optionally containing one or more further heteroatoms.

In some preferred embodiments, R′—O— is C₂ alkoxy, preferably ethoxy.

In some preferred embodiments, R′—O— is C₁ alkoxy, optionally containing one or more further heteroatoms.

In some preferred embodiments, R′—O— is C₁ alkoxy, preferably methoxy.

In some embodiments, R is a straight or branched alkyl, optionally containing one or more rings, optionally containing one or more heteroatoms, and/or optionally containing one or more double bonds; and R′—O— is a straight or branched alkoxy group, optionally containing one or more rings, optionally containing one or more further heteroatoms, and/or optionally containing one or more double bonds.

In some embodiments, R is a straight or branched C₁-C₁₅, C₁-C₁₂, C₁-C₁₀, C₁-C₈, C₁-C₆, or C₁-C₈ alkyl; and R′—O— is a straight or branched, and preferably straight C₁-C₁₀, C₁-C₈, C₁-C₆, C₁-C₅, C₁-C₄, or C₁-C₃ alkoxy.

In some embodiments, R is a straight or branched C₁-C₁₀, C₁-C₈, C₁-C₆, or C₁-C₈ alkyl; and R′—O— is a straight or branched, and preferably straight C₁-C₁₀, C₁-C₈, C₁-C₆, C₁-C₅, C₁-C₄, or C₁-C₃ alkoxy.

In preferred embodiments, R is a straight or branched C₁-C₈ alkyl; and R′—O— is a straight or branched, and preferably straight C₁-C₅, C₁-C₄, or C₁-C₃ alkoxy.

In some preferred embodiments, R is a straight or branched, and preferably branched C₅ alkyl; and R′—O— is a straight or branched, and preferably straight C₁-C₅, C₁-C₄, or C₁-C₃ alkoxy. In a particularly preferred embodiment, R is 1,1-dimethylpropyl; and R′—O— is methoxy.

In some preferred embodiments, R is a straight or branched C₄ alkyl; and R′—O— is a straight or branched, and preferably straight C₁-C₅, C₁-C₄, or C₁-C₃ alkoxy. In some preferred embodiments, R is a straight or branched C₃ alkyl; and R′—O— is a straight or branched, and preferably straight C₁-C₅, C₁-C₄, or C₁-C₃ alkoxy.

In some preferred embodiments, R is a straight or branched C₂ alkyl; and R′—O— is a straight or branched, and preferably straight C₁-C₅, C₁-C₄, or C₁-C₃ alkoxy. In a particularly preferred embodiment, R is ethyl; and R′—O— is n-propoxy.

In some preferred embodiments, R is C₁ alkyl; and R′—O— is a straight or branched, and preferably straight C₁-C₅, C₁-C₄, or C₁-C₃ alkoxy.

In an especially preferred embodiment, the at least one alkoxy alcohol comprises 3-methoxy-3-methyl-1-butanol (MMB).

In preferred embodiments, the at least one alkoxy alcohol comprises a glycol ether.

The at least one glycol ether may preferably comprise an e-series or cellosolve glycol ether.

E-series glycol ethers have the general formula: R′—O—CH₂CH₂OH and are typically produced by a ring-opening reaction between ethylene oxide and an alcohol R′—OH.

In some embodiments, the at least one glycol ether may comprise a p-series glycol ether.

P-series glycol ethers have the general formula R′—O—CH₂CH(CH₃) OH or R′—O—CH(CH₃) CH₂OH, and they are typically produced by a ring-opening reaction between propylene oxide and an alcohol R′—OH.

In preferred embodiments, the p-series glycol ether has the general formula R′—O—CH₂CH(CH₃) OH. In other embodiments, the p-series glycol ether has the general formula R′—O—CH(CH₃) CH₂OH.

The following statements apply to both e-series and p-series glycol ethers.

Statements above relating to the R′—O— group may also be applied to glycol ethers.

In some embodiments, the at least one glycol ether may comprise a diethylene glycol ether and/or dipropylene glycol ether. In a particular embodiment, the at least one glycol ether may comprise diethylene glycol butyl ether (DEGMBE).

In an especially preferred embodiment, the at least one glycol ether comprises propyl cellosolve (2-propoxyethanol). Propyl cellosolve has good physical and chemical parameters and has been shown to be especially effective in mist-based fragrances. Propyl cellosolve is also not classified as an aspiration hazard and as a result allows the composition to pose a greatly reduced respiratory and aspiration risk.

In some embodiments, the at least one alkoxy alcohol comprises at least 5 wt % of the composition, or at least 10, 15, 20, 25, 30, 35, 40, 45, 50, 55, or at least 60 wt % of the composition.

In some embodiments, the at least one alkoxy alcohol comprises no greater than 99 wt % of the composition, or no greater than 98, 97, 96, 95, 94, 93, 92, 91, 90, 85, 80, or no greater than 75 wt % of the composition.

In some embodiments, the at least one alkoxy alcohol comprises between 40-95 wt % of the composition.

The at least one alkoxy alcohol may comprise between 40-90 wt % of the composition, or between 40-85, 40-80, 40-75, 40-70, 40-65, 40-60, 40-55, 40-50, or between 40-45 wt % of the composition.

The at least one alkoxy alcohol may comprise between 45-95 wt % of the composition, or between 50-95, 55-95, 60-95, 65-95, 70-95, 75-95, 80-95, or between 85-95 wt % of the composition.

The at least one alkoxy alcohol may comprise between 45-90 wt % of the composition, or between 50-90, 55-90, 60-90, 65-90, 70-90, 75-90, 80-90, 85-90, 45-85, 50-85, 55-85, 60-85, 65-85, 70-85, 75-85, 80-85, 45-80, 50-80, 55-80, 60-80, 65-80, 70-80, 75-80, 45-75, 50-75, 55-75, 60-75, 65-75, 70-75, 45-70, 50-70, 55-70, 60-70, 65-70, 45-65, 50-65, 55-65, 60-65, 45-60, 50-60, 55-60, 45-55, 50-55, or between 45-50 wt % of the composition.

In preferred embodiments, the at least one alkoxy alcohol comprises at least 40 wt % of the composition, or at least 45, 50, 55, 60, 65, 70, 75, 80, or at least 85 wt % of the composition.

In some embodiments, the composition has a water content of less than 4.5 wt %, or of less than 4, 3.5, 3, 2.5, 2, 1.5, 1, 0.9, 0.8, 0.7, 0.6, 0.5, 0.4, 0.3, 0.2, or of less than 0.1 wt %. In preferred embodiments, the composition contains substantially no water. The composition may consist of entirely organic media. Utilising a lower quantity of water in the composition prevents bacteria and fungi growth, and prevents or reduces immiscibility of the other ingredients, in particular any hydrophobic fragrance oil, that may be in the composition.

In some embodiments, the kinematic viscosity of the composition is less than 15 cP, or less than 14, 13, 12, 11, 10, 9, 8, 7, or less than 6 cP. In preferred embodiments, the kinematic viscosity of the composition is less than 5 cP.

In some embodiments, the density of the composition is less than 1.5 g/cm³, or less than 1.4, 1.3, or 1.2 g/cm³. In preferred embodiments, the density of the composition is less than 1.1 g/cm³, or less than 1.075, 1.05, 1.025, 1, 0.975, or less than 0.95 g/cm³.

In some embodiments, the surface tension of the composition is less than 50 mN/m, or less than 45, or less than 40 mN/m. In preferred embodiments, the surface tension of the composition is less than 35 mN/m, or less than 34, 33, 32, 31, 30, 29, 28, 27, 26, 25, 24, 23, 22, 21, 20, 19, 18, 17, 16, 15, 14, 13, 12, 11, 10, 9, 8, 7, 6, or less than 5 mN/m.

In some preferred embodiments, the kinematic viscosity of the composition is less than 5 cP, the density of the composition is less than 1.1 g/cm³, and the surface tension of the composition is less than 35 mN/m.

Compositions with kinematic viscosities, densities and surface tensions as described in the above paragraphs are particularly well-suited to use as mist-based fragrances. They allow for an improved spray rate and lower fallout.

In some embodiments, the at least one co-solvent comprises at least 0.1 wt % of the composition, or at least 0.2, 0.3, 0.4, 0.5, 1, 1.5, 2, 2.5, 3, 3.5, 4, 4.5, 5, 5.5, 6, 6.5, 7, 7.5, 8, 8.5, 9, 9.5, or at least 10 wt % of the composition.

In some embodiments, the at least one co-solvent comprises no greater than 70 wt % of the composition, or no greater than 65, 60, 55, 50, 45, 40, 35, 30, 25, 20, or no greater than 15 wt % of the composition.

In some embodiments the at least one co-solvent comprises at least 3, 4 or at least 5 wt % of the composition.

In some embodiments, the at least one co-solvent comprises between 5-50 wt % of the composition.

The at least one co-solvent may comprise between 5-45 wt % of the composition, or between 5-40, 5-35, 5-30, 5-25, 5-20, 5-15, or between 5-10 wt % of the composition. The at least one co-solvent may comprise between 10-50 wt % of the composition, or between 15-50, 20-50, 25-50, 30-50, 35-50, 40-50, or between 45-50 wt % of the composition.

The at least one co-solvent may comprise between 10-45 wt % of the composition, or between 15-45, 20-45, 25-45, 30-45, 35-45, 40-45, 10-40, 15-40, 20-40, 25-40, 30-40, 35-40, 10-35, 15-35, 20-35, 25-35, 30-35, 10-30, 15-30, 20-30, 25-30, 10-25, 15-25, 20-25, 10-20, 15-20, or between 10-15 wt % of the composition.

In preferred embodiments, the at least one co-solvent comprises between 5-40 wt.

In some embodiments, the at least one co-solvent comprises a co-solvent selected from the group comprising: glycerol ketal derivates, isoparaffinic hydrocarbons, dialkylene glycol ethers, and combinations thereof.

Glycerol ketal derivates may, in some embodiments, be selected from the group comprising: 2,2-dimethyl-1,3-dioxolane-4-methanol, 2,2-diisobutyl-1,3-dioxolane-4-methanol, 2-isobutyl-2-methyl-1,3-dioxolane-4-methanol, 2-isopropyl-1,3-dioxolane-4-methanol, 2-butyl2-ethyl-1,3-dioxolane-4-methanol, 2-phenyl-1,3-dioxolane-4-methanol and 2-methyl-2-phenyl-1,3-dioxolane-4-methanol, 2,2-dimethyl-1,3-dioxolane-4-acetate, (2-(heptan-3-yl)-1,3-dioxolan-4-yl) methanol and combinations thereof.

Commercial examples of useful isoparaffinic hydrocarbons include the Isopar® class of products from Exxon Mobil, and particularly Isopar M®. In some embodiments, isoparaffinic hydrocarbons may be present in low amounts in the composition, such as below 15 wt % or below 10 wt %. In other embodiments, the composition may contain no isoparaffinic hydrocarbons. Using reduced amounts of isoparaffinic hydrocarbons may be advantageous for further reducing the aspiration hazard posed by the composition.

Dialkylene glycol ethers may be selected from the group comprising: dipropylene glycol monomethyl ether (DPM), dipropylene glycol mono-n-propyl ether (DPNP), dipropylene glycol mono-tert-butyl ether (DPTB), dipropylene glycol mono-n-butyl ether, dipropylene glycol monohexyl ether, diethyleneglycol n-butyl ether (Butyl Diglycol-Ether-BDG), diethylene glycol hexyl ether, diethylene glycol octyl ether, and combinations thereof. In preferred embodiments, the dialkylene glycol ether is dipropylene glycol monomethyl ether (DPM).

In some embodiments, the at least one co-solvent may be selected from the group comprising: dipropylene glycol methyl ether; propylene glycol methyl ether; dipropylene glycol; propylene glycol; pentylene glycol; caprylyl glycol; 1,2-hexanediol; propylene glycol methyl ether acetate; dipropylene glycol methyl ether acetate; propylene glycol monopropyl ether; ethylene glycol mono n-propyl ether; propylene glycol monoethyl ether; diethylene glycol monobutyl ether; ethylene glycol monohexyl ether; dimethoxymethane; dimethylsulfoxide, acetonitrile; ethanol; isopropanol; acetone;

dipropylene glycol monomethyl ether (DPM); Isopar M®; benzyl acetate; 3,5,5-trimethylhexyl acetate; propylene glycol diacetate (PGDA); and combinations thereof.

In preferred embodiments, the at least one co-solvent is selected from the group comprising: dipropylene glycol monomethyl ether (DPM), Isopar M®, 3,5,5-trimethylhexyl acetate, propylene glycol diacetate (PGDA), and combinations thereof.

In some embodiments, the composition contains a single co-solvent. In other embodiments, the composition may contain 2, 3, 4, or 5 different co-solvents.

In some embodiments, the at least one fragrance comprises at least 0.01 wt % of the composition, or at least 0.02, 0.03, 0.04, 0.05, 0.1, 0.2, 0.3, 0.4, 0.5, 1, 1.5, 2, 2.5, 3, 3.5, 4, 4.5, 5, 5.5, 6, 6.5, 7, 7.5, 8, 8.5, 9, 9.5, or at least 10 wt % of the composition.

In some embodiments, the at least one fragrance comprises no greater than 60 wt % of the composition, or no greater than 55, 50, 45, 40, 35, 30, 25, 20, or of no greater than 15 wt % of the composition.

In some embodiments, the at least one fragrance comprises between 2-30 wt % of the composition.

The at least one fragrance may comprise between 2-28 wt % of the composition, or between 2-26, 2-24, 2-22, 2-20, 2-18, 2-16, 2-14, 2-12, 2-10, 2-8, 2-6, or between 2-4 wt % of the composition.

The at least one fragrance may comprise between 4-30 wt % of the composition, or between 6-30, 8-30, 10-30, 12-30, 14-30, 16-30, 18-30, 20-30, 22-30, 24-30, 26-30, or between 28-30 wt % of the composition.

The at least one fragrance may comprise between 4-28 wt % of the composition, or between 6-28, 8-28, 10-28, 12-28, 14-28, 16-28, 18-28, 20-28, 22-28, 24-28, 26-28, 4-26, 6-26, 8-26, 10-26, 12-26, 14-26, 16-26, 18-26, 20-26, 22-26, 24-26, 4-24, 6-24, 8-24, 10-24, 12-24, 14-24, 16-24, 18-24, 20-24, 22-24, 4-22, 6-22, 8-22, 10-22, 12-22, 14-22, 16-22, 18-22, 20-22, 4-20, 6-20, 8-20, 10-20, 12-20, 14-20, 16-20, 18-20, 4-18, 6-18, 8-18, 10-18, 12-18, 14-18, 16-18, 4-16, 6-16, 8-16, 10-16, 12-16, 14-16, 4-14, 6-14, 8-14, 10-14, 12-14, 4-12, 6-12, 8-12, 10-12, 4-10, 6-10, 8-10, 4-8, 6-8, or between 4-6 wt % of the composition.

In preferred embodiments, the at least one fragrance may comprise between 4-25 wt % of the composition, or between 6-20 wt %, or between 6-15 wt %, or between 6-12 wt % of the composition.

Many types of fragrances can be employed in the present composition. Suitable fragrances include but are not limited to fruits such as almond, apple, cherry, grape, pear, pineapple, orange, strawberry, raspberry; musk, flower scents such as lavender-like, roselike, iris-like, carnation-like. Other pleasant scents include herbal and woodland scents derived from pine, spruce and other forest smells. Fragrances may also be derived from various oils, such as essential oils, or from plant materials such as peppermint, spearmint and the like.

The at least one fragrance may comprise one or more fragrances from those listed in European Patent Application EP 1 707 185 A1.

Another source of suitable fragrances is found in Perfumes, Cosmetics and Soaps, Second Edition, edited by W. A. Poucher, 1959.

According to a second aspect of the invention, there is provided a fragrance composition comprising at least one fragrance, at least one alkoxy alcohol solvent, and at least one co-solvent, wherein the kinematic viscosity of the composition is less than 5 cP.

Statements of invention relating to the composition of the first aspect of the invention may be applied mutatis mutandis to the second aspect of the invention.

In preferred embodiments, the kinematic viscosity of the composition is less than 5 cP and the density of the composition is less than 1.1 g/cm³.

In preferred embodiments, the kinematic viscosity of the composition is less than 5 cP and the composition has a surface tension of less than 35 mN/m.

More preferably, the kinematic viscosity of the composition is less than 5 cP, the density of the composition is less than 1.1 g/cm³, and the composition has a surface tension of less than 35 mN/m.

According to a third aspect of the invention, there is provided a method of manufacturing a fragrance composition of the first aspect of the invention, the method comprising the step of combining at least one fragrance, at least one alkoxy alcohol solvent, and at least one co-solvent, and wherein less than 5 wt. % of water is added to or forms part of the composition during manufacture.

Statements of invention above relating to the fragrance composition of the first aspect of the invention may also be applied to the third aspect of the invention.

According to a fourth aspect of the invention, there is provided a method of manufacturing a fragrance composition of the second aspect of the invention, the method comprising the step of combining at least one fragrance, at least one alkoxy alcohol solvent, and at least one co-solvent, and wherein the kinematic viscosity of the composition is less than 5 cP.

Statements of invention above relating to the fragrance compositions of the first and second aspects of the invention may also be applied mutatis mutandis to the fourth aspect of the invention.

According to a fifth aspect of the invention, there is provided an atomiser for dispensing a fragrance; the atomiser comprising:
1. An outer housing;
2. An orifice plate and piezo electric element;
3. A liquid reservoir comprising a liquid to be atomised, the liquid comprising the fragrance composition of the first or second aspect of the invention; and
4. A liquid transport member,
   wherein the liquid transport member extends from within the liquid reservoir to abut the orifice plate, and
   wherein the liquid transport member is substantially contained within the liquid reservoir and both the liquid transport member and liquid reservoir are readily detachable together from the atomiser.

Statements of invention above relating to the fragrance compositions of the first and second aspects of the invention may also be applied mutatis mutandis to the fifth aspect of the invention.

According to a sixth aspect of the invention, there is provided an atomiser for dispensing a fragrance; the atomiser comprising:
a. An outer housing;
b. An orifice plate and piezo electric element;
c. A liquid reservoir comprising a liquid to be atomised, the liquid comprising a fragrance composition comprising at least one fragrance, at least one alkoxy alcohol solvent, and at least one co-solvent; and
d. A liquid transport member,
   wherein the liquid transport member extends from within the liquid reservoir to abut the orifice plate, and
   wherein the liquid transport member is substantially contained within the liquid reservoir and both the liquid transport member and liquid reservoir are readily detachable together from the atomiser.

Statements of invention above relating to the fragrance compositions of the first and second aspects of the invention may also be applied mutatis mutandis to the sixth aspect of the invention.

The following statements apply to the fifth and sixth aspects of the invention.

The fragrance composition may comprise at least 70 wt % of the liquid to be atomised, or at least 75, 80, 85, 90, or at least 95 wt %, or substantially 100 wt % of the liquid to be atomised.

The liquid transport member may be made from any suitable material. Preferably the liquid transport member moves liquid from the reservoir to the plate by wicking or capillary action.

Preferably the liquid transport member of the present invention is a wick comprising at least one material selected from the group comprising paper, cotton, nylon, and polypropylene. Most preferably, the wick is formed from polypropylene.

The wick may be between 4 cm and 14 cm in length, preferably between 5 cm and 12 cm and more preferably between 6 and 10 cm in length. Preferably, the atomiser is powered by battery. Preferably, the atomiser comprises at least one battery, and more preferably at least two batteries.

The atomiser may require between 1 and 12 volts to operate, more preferably 3-4.5 volts. The atomiser may be provided with control circuitry to allow for regular activation of the device over time. Settings may be provided to control length of activation and time between activations. This allows the device to be left in the home or office and provide a constant release of fragrance over many days and weeks.

Activations of the orifice plate to release atomised liquid fragrance composition may last between 1 second and 30 seconds, preferably between 2 seconds and 15 seconds, more preferably between 3 seconds and 10 seconds.

Timing between activations may be between 2 mins and 30 mins, preferably between 5 mins and 20 mins, most preferably between 7 mins and 15 mins.

The skilled person can vary these parameters depending on the intensity of the fragrance desired, strength of fragrance used and concentration of the fragrance composition within the fragrance liquid.

According to a seventh aspect of the invention, there is provided a method of scenting the air comprising activating the atomiser of the fifth or sixth aspect of the invention.

Statements of invention above relating to the atomiser of the fifth or sixth aspect of the invention may also be applied to the seventh aspect of the invention.

The following statements apply to the seventh aspect of the invention.

The method may be performed manually or automatically by setting up dispensing parameters. Automatic dispensing may require control circuits to activate the device at intervals.

According to an eighth aspect of the invention, there is provided a refill of fragrance for use as the liquid reservoir with the atomiser of the fifth aspect of the invention, the refill comprising:
a. A container comprising a liquid comprising the fragrance composition of the first or second aspect of the invention; and
b. A wick extending from the inside of the container to the outside.

Statements of invention above relating to the atomiser of the fifth aspect of the invention may also be applied to the eighth aspect of the invention.

Statements of invention above relating to the fragrance compositions of the first and second aspects of the invention may also be applied mutatis mutandis to the eighth aspect of the invention.

According to a ninth aspect of the invention, there is provided a refill of fragrance for use as the liquid reservoir with the atomiser of the sixth aspect of the invention, the refill comprising:
a. A container comprising a liquid comprising a fragrance composition comprising at least one fragrance, at least one alkoxy alcohol solvent, and at least one co-solvent; and
b. A wick extending from the inside of the container to the outside.

Statements of invention above relating to the atomiser of the sixth aspect of the invention may also be applied to the ninth aspect of the invention.

Statements of invention above relating to the fragrance compositions of the first and second aspects of the invention may also be applied mutatis mutandis to the ninth aspect of the invention

The following statements apply to the eighth and ninth aspects of the invention.

The fragrance composition may comprise at least 70 wt % of the liquid, or at least 75, 80, 85, 90, or at least 95 wt %, or substantially 100 wt % of the liquid.

The refill may be suitable for standalone sale.

Preferably, the container may be a small flask or bottle. This may be made of any material, and preferably glass or plastic.

The container will preferably be readily insertable into the atomiser device of the present invention. This should be such that the user may easily remove empty refills. The user may also decide to replace a partially used refill for a refill of a differing fragrance, if desired.

Including the wick with the refills prevents the mixing of fragrances in the wick. A rapid fragrance change can be carried out without blending different fragrances.

Wicks can get clogged with time. Using a new wick with each refill prevents this from happening.

The wick of the refill may comprise the wick of the atomiser of the fifth aspect of the invention. Statements of invention above relating to the wick of the atomiser of the fifth aspect of the invention may also be applied here. In some preferred embodiments, a polypropylene wick may be used. A polypropylene wick has been shown to yield excellent fragrance dispersion characteristics with virtually no residues remaining.

### DETAILED DESCRIPTION OF THE INVENTION

In order that the invention may be more clearly understood embodiments thereof will now be described, by way of example only.

### Inventive Fragrance Compositions

The following fragrance compositions were prepared:

### Formulation 1:

| | | |
|---|---|---|
| FRAGRANCE | 12 | wt. % |
| DPM (dipropylene glycol methyl ether - co-solvent) | 7 | wt. % |
| 3,5,5-trimethylhexyl acetate - co-solvent | 7 | wt. % |
| ISOPAR M EU (RTM) (isoparaffinic hydrocarbons - co- | 9 | wt. % |
| solvent) | | |
| PROPYL CELLOSOLVE (2-propoxyethanol - glycol ether | 65 | wt. % |
| solvent) | | |

### Formulation 2:

| | | |
|---|---|---|
| FRAGRANCE | 9 | wt. % |
| Acetate-based - co-solvent | 34 | wt. % |
| 3,5,5-trimethylhexyl acetate - co-solvent | 7 | wt. % |
| PROPYL CELLOSOLVE (2-propoxyethanol - glycol ether | 50 | wt. % |
| solvent) | | |

### Formulation 3:

| | | |
|---|---|---|
| FRAGRANCE | 9 | wt. % |
| 3,5,5-trimethylhexyl acetate - co-solvent | 9 | wt. % |
| ISOPAR M EU (RTM) (isoparaffinic hydrocarbons - co- | 9 | wt. % |
| solvent) | | |
| PROPYL CELLOSOLVE (2-propoxyethanol - glycol ether | 70 | wt. % |
| solvent) | | |

### Formulation 4:

| | | |
|---|---|---|
| FRAGRANCE | 9 | wt. % |
| 3,5,5-trimethylhexyl acetate - co-solvent | 20 | wt. % |
| ISOPAR M EU (RTM) (isoparaffinic hydrocarbons - co- | 9 | wt. % |
| solvent) | | |
| PROPYL CELLOSOLVE (2-propoxyethanol - glycol ether | 62 | wt. % |
| solvent) | | |

### Formulation 5:

| | | |
|---|---|---|
| FRAGRANCE | 12 | wt. % |
| Benzyl acetate co-solvent | 9 | wt. % |
| ISOPAR M EU (RTM) (isoparaffinic hydrocarbons - co- | 9 | wt. % |
| solvent) | | |
| MMB (methoxy-3-methyl-1-butanol solvent) | 62 | wt. % |

### Fragrance Testing

Formulations 1-5 were independently loaded into a piezoelectric Air Wick® Essential Mist Diffuser (set on max settings) and independently used to fragrance the insides of small or large testing booths prior to olfactory assessment by a tester.

Insides of small and large booths were respectively fragranced for 10 minutes or 1 hour prior to assessment.

Results are displayed below:

Formulation 1 (small booth): Fragrance displayed good character and strength. No off-putting notes.

Formulation 2 (large booth): Fragrance displayed good character and strength.

Formulation 3 (small booth): Fragrance displayed good strength. No bad solvent notes detected.

Formulation 4 (large booth): Fragrance displayed good character and strength.

Formulation 5 (small booth): Fragrance displayed good character and strength with no off notes.

All formulations worked particularly well in the piezo electric diffuser, enabling efficient diffusion of the formulations throughout the airspace.

The above embodiments are described by way of example only. Many variations are possible without departing from the scope of the invention as defined in the appended claims.

1. A fragrance composition comprising at least one fragrance, at least one alkoxy alcohol solvent, and at least one co-solvent, and wherein the composition has a water content of less than 5 wt. %.

2. The fragrance composition of claim 1, wherein the at least one alkoxy alcohol solvent contains a chain of 1-10 carbon atoms, and preferably 1-5 carbon atoms directly connecting the oxygen atom of the alkoxy group to the oxygen atom of the alcohol group.

3. The fragrance composition of claim 1, wherein the at least one alkoxy alcohol solvent comprises a glycol ether.

4. The fragrance composition of claim 3, wherein the at least one glycol ether comprises an e-series or cellosolve glycol ether.

5. The fragrance composition of claim 4, wherein the at least one glycol ether comprises propyl cellosolve.

6. The fragrance composition of claim 1, wherein the at least one alkoxy alcohol solvent comprises a compound of Formula (I):
R′—O—ROH (I)
wherein
R′—O— is: a straight or branched C₁-C₁₀ alkoxy, preferably C₁-C₅ alkoxy group, optionally containing one or more rings, optionally containing one or more further heteroatoms, and/or optionally containing one or more double bonds; an aryloxy group; or an arylalkoxy group; and
R is: a straight or branched C₁-C₁₅ alkyl, preferably C₁-C₁₀ alkyl, optionally containing one or more rings, optionally containing one or more heteroatoms, and/or optionally containing one or more double bonds; an aryl; or an aralkyl.

7. The fragrance composition of claim 6,
wherein
R is: a straight or branched C₁-C₅ alkyl; and
R′—O— is: a straight or branched, and preferably straight C₁-C₅, C₁-C₄, or C₁-C₃ alkoxy.

8. The fragrance composition of claim 7, wherein the at least one alkoxy alcohol solvent comprises 3-methoxy-3-methyl-1-butanol.

9. The fragrance composition of claim 1, wherein the at least one alkoxy alcohol solvent comprises between 40-90 wt % of the composition.

10. The fragrance composition of claim 1, wherein the composition contains substantially no water.

11. The fragrance composition of claim 1, wherein the viscosity of the composition is less than 5 cP.

12. The fragrance composition of claim 11, wherein the density of the composition is less than 1.1 g/cm³.

13. The fragrance composition of claim 1, wherein the composition has a surface tension of less than 35 mN/m.

14. The fragrance composition of claim 1, wherein the at least one co-solvent comprises between 5-50 wt % of the composition.

15. The fragrance composition of claim 1, wherein the at least one co-solvent is selected from the group comprising: dipropylene glycol monomethyl ether (DPM), ISOPAR M, 3,5,5-trimethylhexyl acetate, propylene glycol diacetate (PGDA), and combinations thereof.

16. The fragrance composition of claim 1, wherein the at least one fragrance comprises between 2-30 wt % of the fragrance composition.

17. An atomiser dispensing a fragrance; the atomiser comprising:
a. an outer housing;
b. an orifice plate and piezo electric element;
c. a liquid reservoir comprising a liquid to be atomised, the liquid comprising the fragrance composition of claim 1; and
d. a liquid transport member,
wherein the liquid transport member extends from within the liquid reservoir to abut the orifice plate, and
wherein the liquid transport member is substantially contained within the liquid reservoir and
both the liquid transport member and liquid reservoir are readily detachable together from the atomiser.

18. A method of scenting the air comprising activating the atomiser of claim 17.

19. A refill of fragrance as the liquid reservoir with the atomiser of claim 17, the refill comprising:
a. a container comprising a liquid comprising the fragrance composition of claim 1; and
b. A wick extending from the inside of the container to the outside.
